(19) United States
(12) Patent Application Publication
(10) Pub. No.: US 2003/0180351 A1
(43) Pub. Date: Sep. 25, 2003

(54) Pharmaceutically active composition and dispensing device

(76) Inventors: Reinhard Gluck, Spiegel Bern (CH); Ulrich Gluck, Meggen/Luzern (CH); Andre Collioud, Jegenstorf (CH)

Correspondence Address: Judson K. Champlin, WESTMAN CHAMPLIN & KELLY, International Centre - Suite 1600, 900 South Second Avenue, Minneapolis, MN, 55402-3319, US

(21) Appl. No.: 10/385,306

(22) Filed: Mar. 10, 2003

Related U.S. Application Data
(63) Continuation of application No. 09/831,126, filed on Sep. 24, 2001, now abandoned.
Application No. 09/831,126 is a 371 of international application No. PCT/EP99/08557, filed on Nov. 8, 1999.

(30) Foreign Application Priority Data
Nov. 6, 1998 (DE) 198 51 282.1

(51) Int. Cl. A61K 9/127; A61M 11/00

(52) U.S. Cl. 424/450; 128/200.22

(57) ABSTRACT

The invention relates to an improved pharmaceutical substance as well as to an improved application device which is suitable for a more effective nasal administration of a substance.

The invention cites various documents. The subject matter of these documents is herewith incorporated into the specification by reference.

The present invention relates to compositions of a pharmaceutically active substance, in particular antigens that are mixed with a mucosal adjuvant and that may be applied to the nasal mucosa by means of an application device in form of a spray applicator in such a way that an effectiveness may be achieved that has never been achieved before.

In particular, the application comprises a vaccine for intranasal application consisting of:

(a) influenza surface proteins which are formulated in a liposomal way (virosomes);

(b) a mucosal adjuvant of bacterial origin;

(c) a specific spray applicator which is constructed in such a way that almost 100% of a sprayed dose can be fully applied to the nasal mucosa that is important with regard to effectiveness.

Preferably, the antigens are influenza surface glycoproteins that are formulated in a liposomal way (so-called virosomes). These, virosomes are mixed with a mucosal adjuvant of bacterial origin. Ideally, it is derived from the class of active or inactive toxins such as heat labile toxin (HLT), cholera toxin (CT) or procholera genoid (PCG). The nasal spray applicator is constructed in such a way that the effective substance can almost be fully applied to the nasal mucosa. The formula underlying the invention can be used for various medical indications such as influenza virus vaccination or other infectious diseases as well as for therapeutic treatment of a blocked nose, reconstruction of injured nasal mucosa or general diseases located in the mucosa.

Various authors have described the immunostimulating effect of liposomes (artificial membranes) (Gregoriadis, G.: Immunological adjuvants: A role for liposomes. Immunol. Today 11 (1990) 89-97). This immunostimulating effect can be achieved by coupling the antigens to the surface of the liposomes (Glück, R., Mischler, R., Finkel, B. Que, J. U., Scarpa, B., Cryz, S. J. Jr.: Immunogenicity of new virosome influenza vaccine in the elderly people. Lancet 344 (1994) 160-163). by enclosing the antigens in the inside of the liposomes (Gregoriadis, G., Davis, A., Davis D.: Liposomes as immunological adjuvants: Antigen incorporation studies. Vaccine 5 (1987) 145-151) or simply by mixing them with liposomes (De Haan, A., Geerlings, H. J., Huckshorn, J. P., Van Scharrenburg, G. J. M., Palache, A. M., Wilschut, J.: Mucosal immunoadjuvant activity of liposomes: induction of systemic IgG and secretory IgA responses in mice by intranasal immunization with an influenza subunit vaccine and coadministered liposomes. Vaccine 13 (1995) 613-616). An additional immunostimulating effect was observed when these liposomes were “spiked” with transmembrane and fusogenic glycoproteins (e.g. influenza glycoproteins (Glück, R., Mischler, R., Brantschen, S., Just, M., Althaus, B., Cryz, S. J., Jr.: Immunopotentiating reconstituted influenza virosome (IRIV) vaccine delivery system for immunization against hepatitis A. J. Clin. Invest. 90 (1992) 2491-2495) or Sendai glycoproteins (Gould Fogerite, S. Mazurkiewicz, J. E., Bhisitkul, D., Mannino, R. J.: The reconstitution of biologically active glycoproteins into large liposomes. Use as a delivery vehicle to animal cells. In: C. H. Kim, J. Diwan, H. Tedeschi and J. J. Salerno (ed.), Advances in Membrane Biochemistry and Bioenergetics, Plenum Publishing Corp., New York (1987) p. 569).

However, this effect has mostly been described after parenteral application (i.m., s.c. or i.p.) up to now. Some authors, however, found that such formulations can be successfully applied with conventional methods (droppers or conventional nasal sprays). Yet, the results are almost exclusively based on experiments with test animals, mostly mice, which have a nasal mucosa that is, in relation to their weight, much larger than the nasal mucosa of humans. Furthermore, the amount of antigens given was so high that the use in humans would be out of the question for economic reasons alone, but also for reasons of product safety. Moreover, it could be shown that, if the dosage of the antigen (influenza) was reasonable, no satisfactory effect could be achieved in spite of liposomal formulation and correct nasal application.

Therefore, other authors tried to develop an effective vaccine that can be applied nasally by mixing the antigens with a mucosal adjuvant of bacterial origin instead of the liposomal formulation. In this case, in particular HLT, CT or non-toxic derivatives of HLT or CT (Elson, C. O., Ealding, W.: Generalized systemic and mucosal immunity in mice after mucosal stimulation with cholera toxin. J. Immunol. 132 (1984) 2736-2741) were used.

The results in the literature in fact showed promising effects after nasal application, these effects having been achieved again mainly with test animals. However, few clinical test with humans confirmed a certain effectiveness (Tamura, S. -J., Ishihira, K., Aizawa, C., Kurata, T.: Mechanism of enhancement of the immune responses to influenza vaccine with cholera toxin B subunit and a trace amount of holotoxin. Vaccine 13 (1995) 339-341). Yet again, the amount of antigens and adjuvant used was very high, which did not dispel doubts as to product safety and the possibility of commercialisation.

The unsatisfactory applicator systems that are at disposal today are further disadvantages of the tests described so far as to the development of a nasally applicable, preventive or therapeutically effective vaccine.

At the moment, nasal sprays are available as mono-, bi- and multi-dose applicators. Comprehensive examinations and tests of the-previous nabal sprays by means of dye application (methylene blue) and nasal endoscopy showed that only 25%, at the most, of the substance to be sprayed (vaccine, pharmaceutical solution) reaches the mucosa of the mucosa-associated immune system of the turbinates.

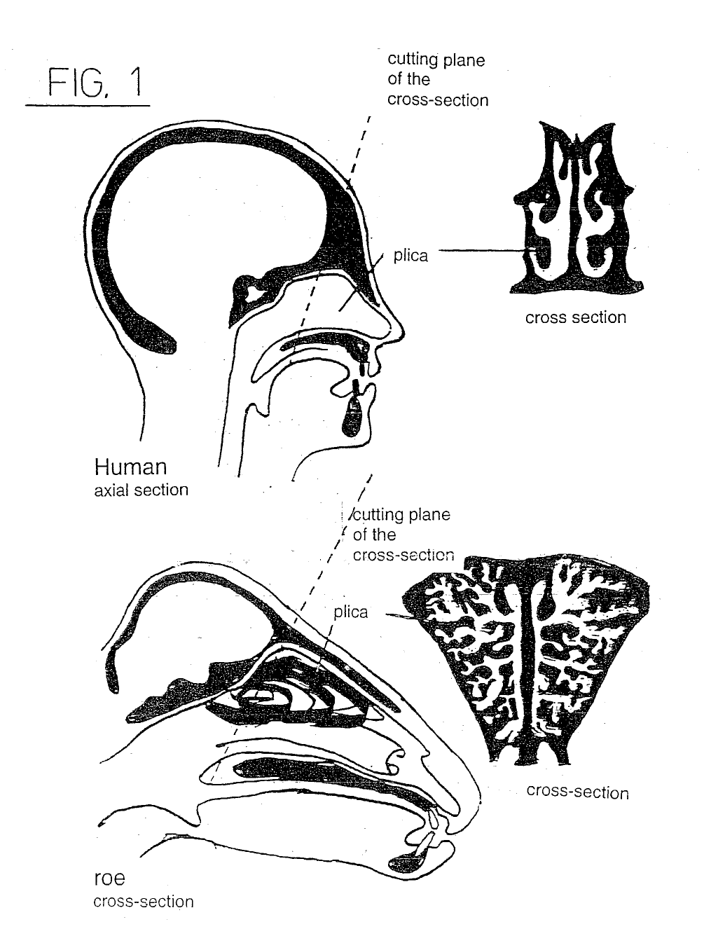

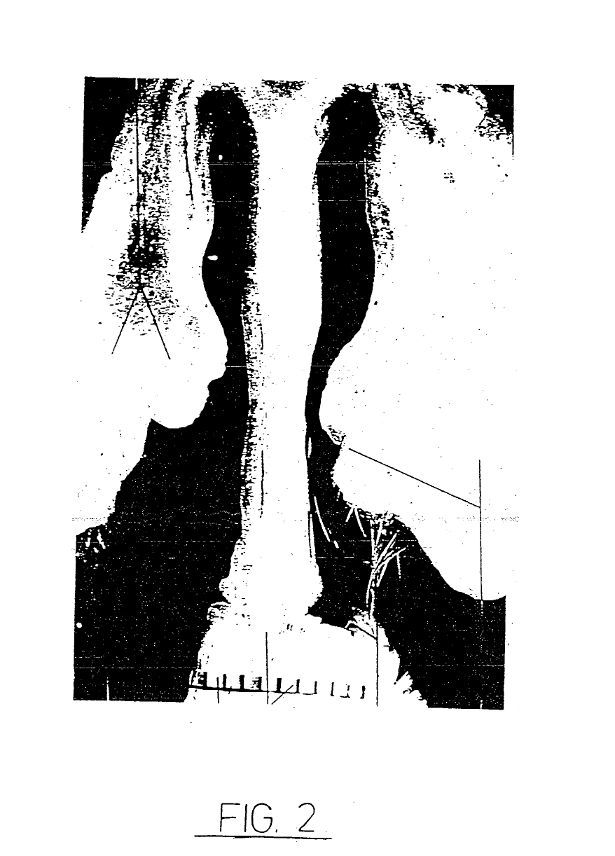

The mucosa of the mucosa-associated immune system is in the nasal cavity at the lateral nasal wall in the region of the turbinates (nasal concha), as is shown, for instance, in FIG. 1 which depicts the human turbinates and the turbinates of a roe. Our examinations have shown that, due to considerable loss in the nasal vestibule (Vestibulum nasi) (cf. FIG. 2) as well as at the nasal septum, only an insufficient amount of the pharmaceutically active substance reaches the main nasal cavities. These nasal structures are lined with immunologically inactive squamous epithelium and sebaceous glands (Walter Becker, Hans-Heinz Naumann, Carl Rudolf Pfaltz: Hals-Nasen-Ohren-Heilkunde, Thieme Verlag, Stuttgart, New York 1992).

There are various reasons for the fact that it has not been possible so far to spray a sufficient dose on the respiratory mucosa (site of the specific and unspecific defence) with previous nasal sprays:

(1) Due to insufficient knowledge as to anatomy, in the case of spray application, the main vector of the spray stream is directed in the wrong direction. As a result, the pharmaceutically active substance does hardly get to the main nasal cavities. The sprayed fluid partly flows out via the upper lip.

(2) The diameter of the spraying piece of the nose part is not adapted to anatomic conditions. The inner nostril naturally has the form of a gap and has the function of a jet (cf. FIG. 2). The nose pieces that usually are wide cannot overcome this anatomic narrowness. If the liquid is sprayed before the Lumen nasi, only the sector of the spray cone reaches the main nasal cavities which corresponds to the width of the Lumen nasi.

(3) The optimum angle of spraying has not yet been scientifically searched for. As regards conventional spray applicators, this angle arbitrarily varies between 30° and 80° to the horizontal line if the head is held in a normal position or to a vertical line to the longitudinal axis of the human body. As a consequence, the considerable loss, i.e. up to 90% of the volume applied, can be explained. Our systematic analyses have shown that the optimum angle of spraying is in the range of 50° to 80°.

(4) Conventional nasal sprays have too short a nose piece. The distance between the finger collar and the spray opening is too short. With the previous sprays not even the inner nostril can be reached. The nose piece should be at least 0.7 cm longer, preferably the nose piece has a length of 1 to 1.5 cm.

Thus, the technical problem underlying the present invention was to to provide an improved composition of a pharmaceutically active substance as well as an improved application device, in particular for preventing and treating infectious diseases. This technical problem has been solved by providing the embodiments characterised in the claims.

The most effective composition to achieve a preventive and therapeutic effect is the formula of the surprisingly co-stimulating effect of liposomes mixed with a mucosal adjuvant and applied via a novel application device or a novel nasal spray.

Accordingly, the complex of the invention consists of the following components:

(a) an active substance (antigen);

(b) an equilibrated mixture of liposomes and a mucosal adjuvant; and

(c) a novel medicinal device, i.e. a specific application device or a nasal spray.

In particular, the invention relates to vaccines for intranasal application consisting of:

(a) influenza surface proteins which are formulated in a liposomal way (virosomes);

(b) a mucosal adjuvant of bacterial origin; and

(c) a specific spray applicator which is constructed in such a way that almost 100% of a sprayed dose can be fully applied to the nasal mucosa that is important with regard to effectiveness.

Therefore, the active substance relates to antigens, in particular to influenza glycoproteins which can be readily incorporated into artificial membranes (lioposomes) due to their transmembrane domains. However, other antigens may be coupled alone or in combination with the influenza antigens to the surface of the liposomes. Depending on the chemical nature of the antigens, coupling can be carried out spontaneously or by means of chemical cross-linking molecules, as has already been described earlier (Martin, F. J., Papahadiopoulos, D.: Irreversible coupling of immunoglobulin fragments to preformed vesicles. J. Biol. Chem. 257 (1982) 286-288). A DNA plasmid or an RNA plasmid which codes for an antigen and which may be encapsulated into a liposome may also be used.

Within the meaning of the present invention, the term “antigen” comprises both entire molecules and fragments of these molecules which have antigenic properties and/or which can be used for immunization. Furthermore, the term “antigen” comprises molecules and/or fragments of molecules which have an immunostimulating effect.

The influenza surface protein used in the vaccine of the invention preferably comprises hemagglutinin (HA). Moreover, the influenza surface protein may comprise hemagglutinin (HA) in connection/combination with neuraminidase (NA).

In a specific embodiment, the invention relates to a vaccine comprising additional antigens. In particular, the additional antigens may be bound to the surface of the virosomes.

In another preferred embodiment, the application relates to vaccines of the invention which, apart from antigens consisting of (an) influenza surface protein(s), contain additional antigens, preferably of pathogenic organisms. In this case, the pathogenic organism may be a virus, a bacterium, a fungus or a parasite. These organisms comprise various pathogens such as the hepatitis A virus, the hepatitis B virus, the respiratory syncytial virus (Pneumovirus), parainfluenza virus, mumps virus, Mobilli virus, HIV, diphtheria bacillus (Corynebacterium diphtheriae), tetanus bacillus (Clostridium tetani), pneumococci, Haemophilus influenzae, E. coli, Candida albicans, Candida tropicalis, Candida pseudotropicalis, Candida parapsilosis, Candida krusei, Aspergillus species, Trichomonas species, Trypanosoma species, Leishmania species, Toxoplasma gondii, Plasmodium falciparum, Plasmodium vivax, Plasmodium ovale, Plasmodium malariae, Trematoda species and Nematoda species. In particular, the Trematoda species comprise Schistoma haematobium, S. mansoni, S. japoniaum and the Nematoda species comprise Tricharis trichiura, Ascaris lumbricoides and Trichinella spiralis.

In addition, the invention comprises a vaccine that is an influenza vaccine. In particular, the invention comprises a vaccine that can be used for preventing and/or treating general infections/infectious diseases. Furthermore, the vaccine is used for preventing and/or treating influenza diseases, for preventing and/or treating a blocked nose, for treating injuries of the nasal mucosa. Moreover, the invention preferably comprises vaccines with the content of hemagglutinin per dose (100 μl) ranging from 1-30 μg, more preferably from 3-10 μg and most preferably being 3.75 μg.

In a preferred embodiment, the invention relates to a vaccine with the ratio of liposomes-phospholipids to HA ranging from 1:10 to 20:1, more preferably from 1:1 to 10:1 and most preferably the ratio is 3:1.

Furthermore, the invention preferably relates to a vaccine with the phospholipid of the liposomes being selected from the group consisting of neutral, cationic and/or anionic phospholipids.

In a preferred embodiment, the invention relates to a vaccine in which the mucosal adjuvant is an active toxin, an inactive toxin and/or a non-toxic toxin. In particular, the mucosal adjuvant comprises the mucosal adjuvant, preferably heat labile toxin (HLT), cholera toxin (CT) and/or procholera genoid (PCG). In another preferred embodiment, the mucosal adjuvant is the heat labile toxin (HLT) of Escherichia coli. In another preferred embodiment, the toxins may be inactivated. Such inactivation may be achieved using recombination technology.

In another embodiment, the invention comprises a vaccine which comprises heat labile toxin (HLT) and/or cholera toxin (CT) at a ratio ranging from 1:2 to 20:1, more preferably from 1:1 and 1:10 and which most preferably is 7.5:1.

The term “equilibrated mixture of liposomes and a mucosal adjuvant”, which was used earlier, on the one hand relates to the correct ratio of antigen to phospholipids as well as to the mucosal adjuvant, which is necessary for achieving a satisfactory effectiveness. Clinical tests have shown the following: the ideal ratio of phospholipid (e.g. phosphatidyl choline, phosphatidyl ethanolamine, neutral, anionic or cationic phospholipids) to the influenza antigen ranges from 1:1 to 20:1. Most ideally, the ratio is 3:1. The ratio of the influenza antigen to the active mucosal adjuvant (toxin) HLT or CT ranges from 1:2 to 20:1. Most advantageously, the ratio is 7.5:1.

As has been mentioned above, the ratio of the influenza antigen to the inactive mucosal adjuvant (inactive toxin) PCG or a non-toxic derivative of HLT and CT ranges from 3:1 to 1:20, ideally it is 1:2.

Surprisingly, in-vitro experiments for measuring the immunostimulating mucosal effect have shown that mixing the adjuvants liposomes and mucosal adjuvant does not lead to an addition of the effects, but that the addition is increased by a factor of at least five.

The term “novel medicinal device” relates to a spray applicator which is, in particular, suitable for an intranasal immunization (vaccination). It is not sufficient to create a mucosally active substance if the application is bad or insufficient.

The novel device is characterised by the fact that the anatomic features of the nose are completely taken into consideration:

(1) The distance of the finger collar to the spray head is preferred to be 4.0 cm.

(2) The front part of the nose piece consists of an essentially cylindrical member which, for instance, is 5 mm long and has a maximum diameter of 5 mm.

(3) The angle of spraying of the application device described in this invention is preferably 50° to 70° to the horizontal line.

(4) The axis of the main direction of the application device or the spray applicator is preferably determined by a special collar: the collar can preferably be attached to the front part of the nose piece of the application device and is formed in such a way that it can be placed on the upper lip during spraying so that the direction of spraying is essentially directed to the lateral wall of the nasal cavity.

The collar is preferred to be suitable for being placed on the upper lip since the upper lip is quite close to the nose, but the distance is large enough to form an effective lever to achieve an advantageous orientation. In addition, the sensitive tactile sensors of the lip enable the user to check the correct (centric or parallel) position of the collar without looking in the mirror. What is more, the collar may also have a form suitable for being placed on other parts of the head or the body. The narrow nose piece preferably has a minimum length of 0.5 cm, more preferably of 1 cm, the following thicker nose piece preferably has a length of 1 cm, more preferably of 2 cm. The distance between the dorsal finger surface and the tip of the spray should be at least 2 cm, more preferably 2.5 to 3 cm, or the distance between the part for the fingers (collar) and the tip of the spray should be at least 3 cm, preferably 4 to 5 cm. The angle of spraying should preferably be 50° to 70° to the horizontal line if the head is held in a normal vertical position.

Furthermore, the main vector of the sprayed dose should be directed between the middle and the lower nasal,concha, i.e. it should be directed to the middle meatus of the nose. This normally requires an almost horizontal direction of the applicator. As described in the present invention, such a direction may be fixed by attaching a special applicator collar. It is also possible that the collar and the applicator are made in one piece or are integrally connected. In this case, the attaching device/connecting device is fixed tightly to the applicator or the attaching device/connecting device and the applicator are one piece.

In a preferred embodiment, the spray applicator is a device the nose piece and the spray head of which have a maximum thickness of 5 mm and a minimum length of 5 mm, preferably 7 mm, the distance between the collar and the tip of the spray head is at least 3 cm and which may be inserted into the nostril with an attachable collar in such a way that the main sector of the nasal spray forms an angle of approx. 15 to 20° to the horizontal line.

In another preferred embodiment, the antigen is a mixture of influenza surface antigens which is presented on the surface of liposomes. This substance may be combined with antigens of other pathogenic microorganisms. As has already been mentioned earlier, the vaccine of the invention can, thus, contain further antigens, prefer ably antigens of other pathogenic organisms.

In another preferred embodiment, the method described earlier serves the prophylaxis of infectious diseases, the treatment of a blocked nose and various disorders of the nasal mucosa.

In the following, by referring to the Figures, preferred embodiments of the invention are exemplarily described. The Figures show:

FIG. 1: a comparative illustration of human turbinates and turbinates of a roe;

FIG. 2: a schematic illustration of the anatomic structure of an inner nostril;

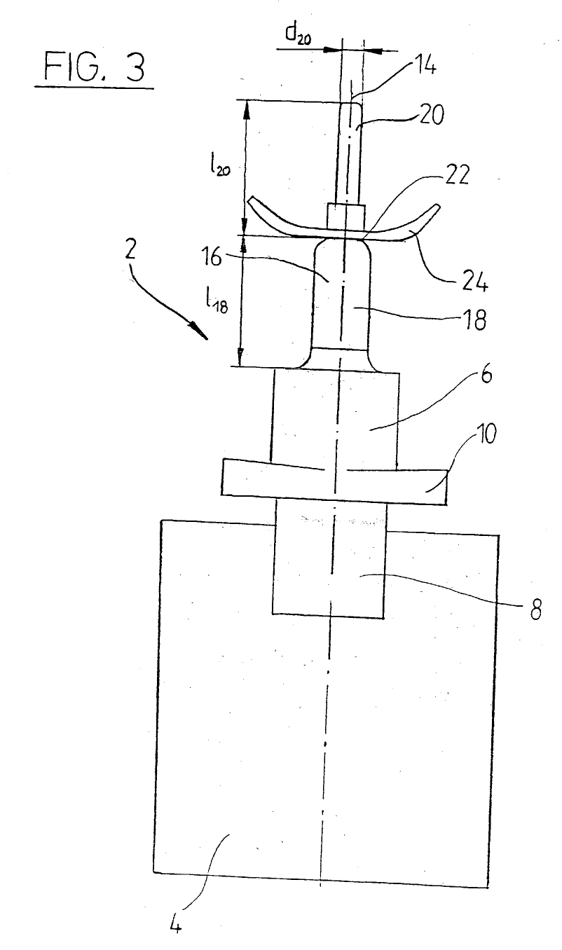

FIG. 3: a schematic illustration of the basic structure of a preferred embodiment of the application device of the invention;

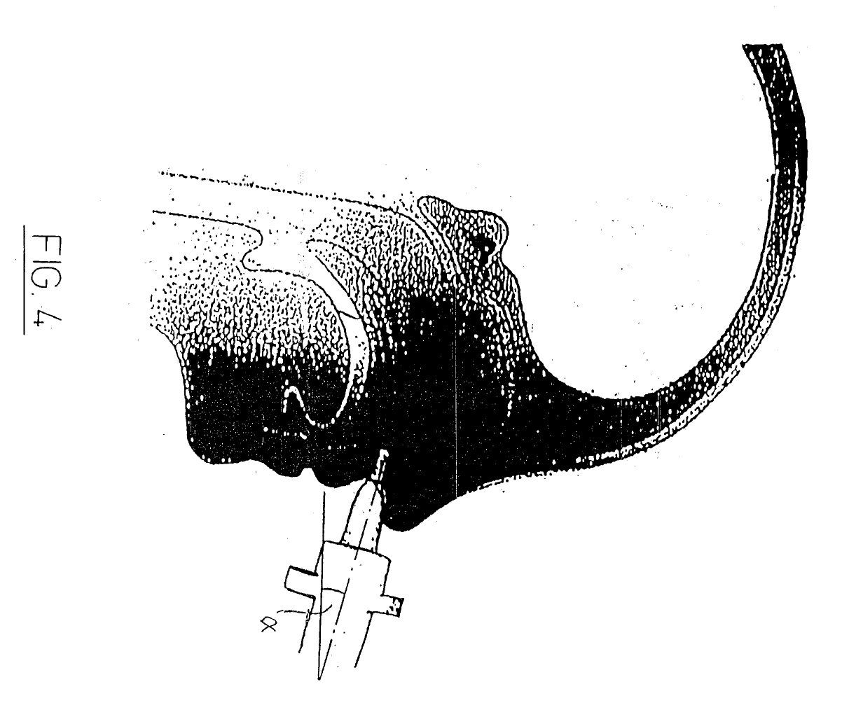

FIG. 4: an illustration of the correct angle of spraying in the case of an effective application;

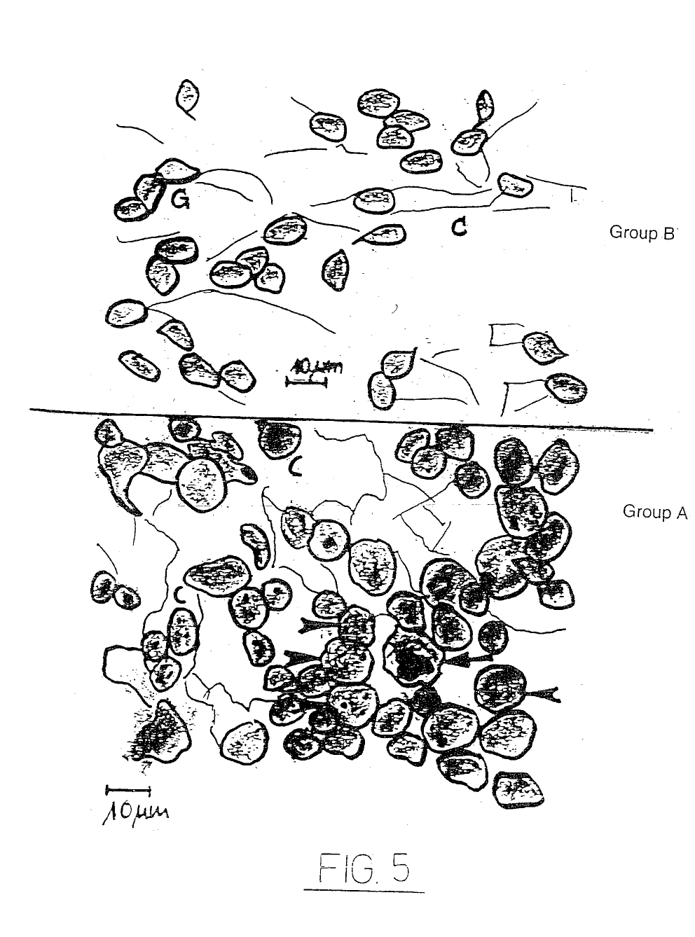

FIG. 5: an illustration of a nasal cytology: quantification of various cell populations which were obtained by nasal smear of test persons (20 of each group) up to 29 days after three different kinds of intranasal vaccination. The significant increase in centroblasts is to be regarded as sign of a local immune response in group A in contrast to the significantly lower increase in groups B and C (p<0.005);

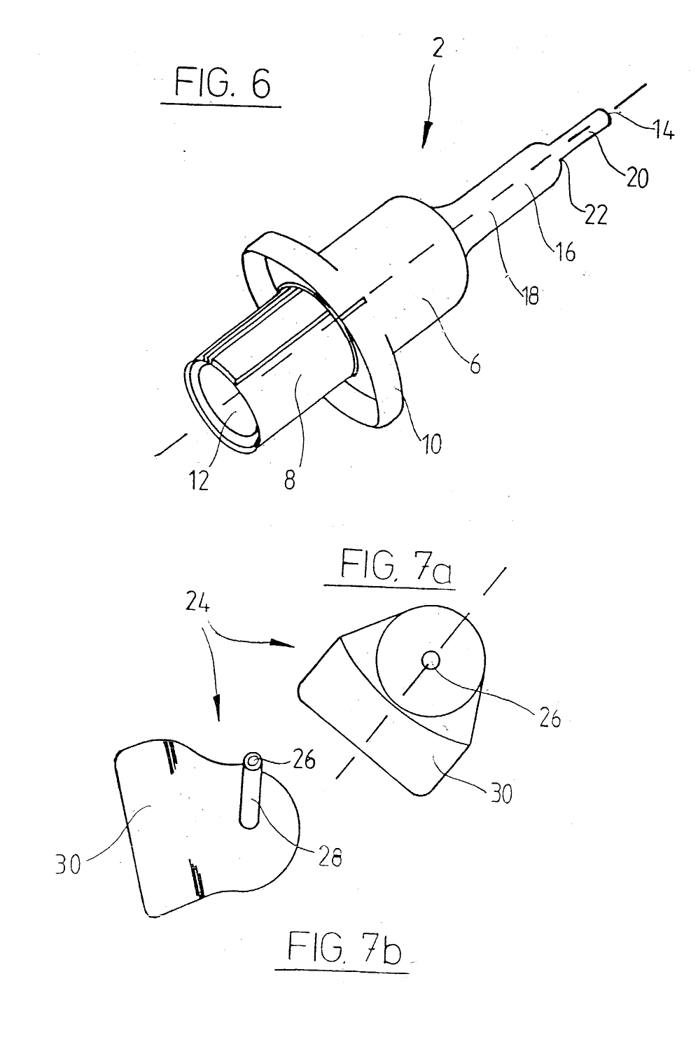

FIG. 6: a preferred embodiment of the application device without collar; and

FIGS. 7a and 7b: two illustrations of a preferred embodiment of a collar for the application device of the invention of FIG. 6.

### EXAMPLE 1

Preparation at Mucosal Virosomal Influenza Virus Vaccine with HLT as Additional Mucosal Adjuvant

The preparation of influenza virosomal vaccines has been described in Glück, R. Wegmann, A.: Liposomal presentation of influenza antigens. In: Nicholson, K. G., Webster, R. G., Hay, A. J., ed., Textbook of Influenza (1998) p. 400-409. London: Blackwell. In brief, the preparation comprises: the strains H1N1 A/Singapore/6/86, H3N2 A/Wuhan/359/95 and B/Beijing/184/93 of the influenza virus, which were cultivated in embryonated hen eggs, were supplied by the National Institute of Biological Standards and Control, London, GB. Intact virions were isolated from the Chorioallantois liquid by means of zonal centrifugation and inactivated with β-propiolactone. The purified virions were added to a buffer which contained 0.1 M octaethylene glycolmono(N-dodecyl)ether (OEG) (Nikko Chemicals, Japan) in PBS-NaCl. They were incubated for 20 min at 21° C. in order to allow complete degradation of the virus components.

For extracting hemagglutinin (HA) and neuraminidase (NA), the mixture was centrifuged for 60 min at 100,000×g. The supernatant containing HA, NA and viral phospholipids (PL) was used for preparing the different intranasal vaccine formulations. Additional phospholipids (phosphatidyl choline [Lipoid, Germany]) were added and made soluble. The virosomes were spontaneously formed during removal of the OEG detergent by chromatography. 0.5 μg heat labile toxin (HLT) of E. coli of the production strain E. coli HE22VK (Vogel, F. R., Powell, M. F.: A compendium of vaccine adjuvants and excipients. In: Vaccine Design: The Subunit and Adjuvant Approach (M. F. Powell, M. J. Newmann, ed.), Plenum Press, New York (1995), p. 141) were added as mucosal adjuvant to a mucosal vaccine dose (100 μl) containing 3.75 μg HA of each of three influenza virus strains, as recommended by the WHO, and 35 μg lecithin.

### EXAMPLE 2

The E. coli cells were precipitated in a passing through centrifuge (Westphalia AG) and suspended in phosphate buffered saline (PBS, 6.06 g/l Na₂HPO₄, 1.46 g/l KH₂PO₄, 2.4 g/l NaCl pH 7.4).

The intracellular heat labile toxin (HLT) was released by disruption of the cells in a ball mill (e.g. by Dyna-Mill, W. Bachofen AG) or in a French press. For example: 10 l cell suspension is pumped through the ball mill at a flow rate of 33 ml/min. The ball mill is filled with 500 ml of glass balls, rotates with 3000 min⁻¹ and the slot has a size of 0.05 mm.

The solid cell components in the cell disruption solution were separated by means of tangential microfiltration. For example: the 10 l cell lysate were concentrated in a Prostak system (Millipore AG) to approx. 4 l using a filter with a pore size of 0.2μ. Then, the mixture is rinsed with 24 l PBS. The permeate is filtered through an 0.2 μ-sterile filter (e.g. Gelman Supor DCF, Pall). The HLT is isolated from the permeate by means of affinity chromatography. Immobilized galactose (galactose gel, e.g. Pierce) or immolized lactose (lactosyl gel, e.g. Parmacia) is used as stationary phase. The mobile phase consists of PBS (buffer A) and 5% (w/v) lactose in semi-concentrated PBS (buffer B). For example: the sterile filtered cell lysate is applied to the column which has been pre-conditioned with buffer A and then the column is rinsed with buffer A until the UV absorption has reached the base line at 280 nm. Subsequently, the HLT is eluted from the column with buffer B.

### EXAMPLE 3

Preparation of a Mucosal Virosomal Influenza Vaccine with PCG as Additional Mucosal Adjuvant

The preparation of an influenza virosomal vaccine has been described in Example 1. 6 μg procholera genoid, prepared by heating purified CT which was derived from V. cholerae (Inaba 569B) were added as mucosal adjuvant (Pierce, N. F., Cray, W. C., Sacci, J. B., Craig, J. P., Germanier, R., Fürer, E.: Procholeragenoid: A safe and effective antigen for oral immunization against experimental cholera. Inf. Immunity 40, 3 (1983) 1112-1118) to a mucosal vaccine dose (100 μl) containing 3.75 μg HA of each of three influenza vaccine strains, as recommended by the WHO, and 35 μg lecithin.

### EXAMPLE 4

Preparation of a Mucosal Hepatitis B (HBs) Vaccine

Phosphatidyl ethanolamine (R. Berchtold, Biochemisches Labor, Bern, Switzerland) was dissolved in methanol and 0.1% (v/v) triethylamine were added. Then, the solution was mixed with γ-maleimidobutyric acid-N-hydroxysuccinimide ester (GMBS) (Pierce Chemical Company, Rockford, Ill.) (ratio of phosphatidyl ethanolamine to, GMBS is 2:1), which has previously been dissolved in dimethylsulfoxide (DMSO). After incubation for 15 min at room temperature, the solvents were vaporized under a vacuum in a Speedvac centrifuge for one hour. Recombinant HBs particle were prepared and purified according to known methods in CHO cell lines.

The particles were treated with 40 mM/l DL-dithiothreitol (DTT) for 5 min at room temperature to obtain reduced HBs particles with free cysteine-residues. The DTT was removed using a Sephadex-G10 column (Pharmacia LKB Biotechnology, Uppsala, Sweden) and octaethylene glycol (Fluka Chemicals, Switzerland) (OEG) was added in a final concentration of 100 mM/l. The vaporized phosphatidyl ethanolamine-GMBS was then mixed with the HBs solution for one hour (ratio HBs: GMBS=1:2), GMBS that was not bound was caught by cysteine. The reactions were monitored by means of thin-layer chromatography.

32 mg phosphatidyl choline (Lipoid GmbH, Ludwigshafen, Germany) and 6 mg phosphatidyl ethanolamine were added to the phosphatidyl ethanolamine-GMBS-HBs, which previously had been cross-linked, and the mixture was dissolved in an overall volume of 2.66 ml PBS containing 100 mM OEG (PBS—OEG).

The influenza A/Singapore hemagglutinin was purified as has been described earlier in Example 1. A solution containing 4 mg hemagglutinin was centrifuged for 30 min at 100,000 g and the precipitate was dissolved in 1.33 ml PBS—OEG.

The phospholipids and the hemagglutinin solution were mixed and subjected to ultrasonication for 1 min. Then, the mixture was centrifuged for 1 hour at 100,000 g and the supernatant was sterile filtered (0.22 μm).

Virosomes with adsorbed HBs were subsequently removed by removal of the detergent using BioRad-SM-Bio-Beads according to Example 1. 10 μg/ml HLT of Example 2 were added to a stock solution containing 50 μg HBs antigen/ml and filled into the spray applicators according to Example 6.

### EXAMPLE 5

In-Vitro Test for Testing the Activity of the Mucosal Adjuvant

The biological activities of the mucosal adjuvants were measured with influenza virosomes, HLT, PCG, a mixture of virosomes and HLT according to Example 1 and a mixture of virosomes and PCG according to Example 2. The following solutions were added to Y-1-adrenal cells ATCC: CCL 79 (Y-1adrenal tumor, mouse steroid secreting) in miniculture according to Sack, D. A. and Sack, R. B. (Sack, D. A. and Sack, R. B.: Test for enterotoxigenic Escherichia coli using Y-1 adrenal cells in miniculture. Infect. Immun. 11 (1974) 334-336):

(a) HLT (5 μg/ml), (b) PCG (60 μg/ml), (c) influenza virosomes and HLT: 37 μg/ml influenza hemagglutinin of three strains (H1N1, H3N2, B), 100 μg/ml phosphatidyl choline and 5 μg/ml as well as (d) influenza virosomes and PCG: 37 μg/ml influenza hemagglutinin of three strains, 100 μg/ml phosphatidyl choline and 60 mg/ml PCG.

The following adjuvant activities (in units) were measured:

(a) 15 units, (b) 6 units, (c) 80 units, (d) 36 units. The highest biological adjuvant activity was achieved with the mixture of virosomes and HLT or virosomes and PCG. The adjuvant effect was determined by means of cell toxicity parameters.

### EXAMPLE 6

Clinical Assessment of Various Spray Applicators

The basic structure of the application device of the invention is essentially comparable to the one of conventional spray applicators, is, however, adapted particularly well to the anatomic conditions for nasal application of any substance, in particular of the pharmaceutically active substance described herein.

FIGS. 3 and 6 show a preferred embodiment of the application device 2 of the invention. In the side view of the application device (spray applicator) 2 shown in FIG. 3, the essential components can be seen. The application device 2 essentially shows a container 4 with a pharmaceutically active substance (not depicted) and a spray or pumping mechanism 6. The pumping mechanism 6 is linked with the container 4 in a fluid-tight manner via a connection section 8. Pumping and spraying with the application device 2 of the invention is done conventionally by pressing a collar 10. The pharmaceutically active substance in the container 4 is released at the outlet 14 via a channel 12 extending through the pump mechanism.

The application device 2 of the invention differs from conventional spray applicators in particular in the structural design of the nose piece 1b which is of great importance as regards the effective application of the pharmaceutically active substance. The nose piece 16 preferably is integrally connected with the pump mechanism 6 and the finger collar 10. The nose piece 16 is preferably divided in two sections 18 and 20, wherein the section 18 located in the direction of the finger collar 10 has a larger diameter than the one of the section 20 of the nose piece 16 located next to the outlet 14. The transition section between the two sections 16 and 20 forms a stop 22 for a collar 24 that may—according to a preferred embodiment of the present invention—be attached to the section 20 of the nose piece 16 for placing the applicator on the user's upper lip.

The section 20 of the nose piece 16, which is located next to the outlet 14, is preferred to have an essentially cylindrical form and preferably has a maximum diameter d₂₀ of 5 mm, more preferably a diameter ranging from 2 to 4 mm. The front section 20 of the nose piece 16 has a length I₂₀ of, for instance, 5 mm, preferably of at least 10 mm and most preferably of between 10 and 20 mm. The collar 24 may preferably be put over the section 20 of the nose piece 16 starting from the outlet 14. Preferably the collar is fixed tightly on the application device 2 by means of a locking means so that it cannot rotate. The locking means may, for example, be realised in form of a polygon profile, e.g. with elliptic cross-section, at the front section 20 of the nose piece 16 and a corresponding recess 26 in the collar 24.

According to another embodiment of the application device 2 of the invention, the section of the spray applicator that is introduced into the nose of a patient may also be fixed to the collar 24. Such an embodiment is exemplarily shown in FIG. 7b. As is shown in FIG. 7b, there is an essentially tubular portion 28 at the collar 24 which may be placed upon the section 20 of the nose piece 16 located next to the outlet 14. In this case, the dimensions of the tubular portion 28 are preferred to be essentially the same as the ones of the aforementioned embodiment (d₂₀ and I₂₀).

The section 18 of the nose piece 16 preferably has a length 118 of at least 10 mm, more preferably of approx. 20 mm. The dimension of the two sections 18 and 20 are preferred to be selected in such a way that the distance between the dorsal surface of the finger of the person using the spray and the tip of the spray or the outlet 14 is at least 2 cm, advantageously 2.5 to 3 cm. This is guaranteed in particular if the distance between the collar 24 and the tip of the spray 14 is at least 3 cm or, advantageously 4 to 5 cm.

If the application device 2 has this dimension, it is possible to spray the pharmaceutically active substance much more exactly into the main nasal cavities or the inner nostril, so that a significantly higher effectiveness compared to conventional systems can be achieved.

The application device 2 of the invention advantageously has a collar 24 so that, in addition to the more favourable size, an optimum angle of spraying α can be adjusted. Independent of the aforementioned spray applicator 2, it is, however, also possible to use the collar 24 also with conventional application devices so that with these conventional spray applicators, too, a significantly improved effectiveness may be achieved due to the fact that the angle of spraying α can be adjusted more exactly.

Thus, it is possible to achieve an improved effectiveness with the application device 2 of the invention either by selecting the dimensions of the nose piece 16 or by using a suitable collar 24 for adjusting the optimum angle of spraying α. However, a combination of the novel application device 2 having the above-described-dimension and the collar 24 for adjusting the optimum angle of spraying α is preferred in order to achieve optimum results in therapy.

As has been explained in brief before, the collar 24 is put over the section 20 of the nasal piece 16 along with the tubular section 28. If the collar 24 is attached to the application device 24, it preferably cannot rotate. The locking means may, for instance, be a circular profile that is flattened on at least one side and provided at the nose piece. With this profile the collar 24 can be fixed by means of an opening having a corresponding form so that it cannot rotate. The collar 24 has a supporting section 30 which can be placed on the user's upper lip so that it determines the angle of spraying α together with the section 20 of the nose piece 16 that is introduced into the nostril of a patient. The optimum angle of spraying α ranges from 50° to 70°. The supporting section 30 of the collar 24 preferably corresponds to the form of the upper, lip, i.e. it is slightly curved, to obtain an optimum contact and supporting area. The collar 24 may be prepared of any suitable material, however, plastic material that can injection mouldes.

For assessing the effectiveness various application devices were tested. 1% methylene blue solution was filled in five different types of nasal applicators:

(a) a spray applicator according to the invention having a directive collar which fixes the direction of the sprayed dose;

(b) a spray applicator according to the invention without a directive collar;

(c) a conventional commercial spray applicator without the narrow nose piece having a length of 2 cm and a diameter of 4 mm with an angle of spraying of 50°;

(d) a spray applicator according to the invention, however, with an angle of spraying of 50°; and last but not least

(e) a spray applicator according to the invention, however with a finger collar which was only 3 cm away from the spray head.

All spray applicators were so-called bidose applicators. Each spray applicator was tested by five test persons under control of a doctor. The test persons sprayed one dose each of methylene blue solution into their own right and left nostrils. The mucosa turbinates were lit and photographed by means of nasal endoscopy. The intensity of the blue colouring as well as the blue coloured area on the turbinates was assessed by means of a scale of 1 to 4. The results of the assessment were as follows (geometric mean):

Group (a): 19 points, group (b): 16 points, group (c): 7 points, group (d): 9 points, group (e): 12 points.

In group (a) only the spray fluid was almost exclusively administered to the nasal mucosa. In group (b), too, a great share of the content of the spray was sprayed onto the mucosa. In the other groups, an important share of the content of the spray was sprayed into the nasal vestibule. There, the spray fluid to be applied cannot have any effect at all.

### EXAMPLE 7

Comparison Between the Clinical Effectiveness of Intranasal Virosomal Influenza Vaccines With and Without HLT Applied in a New Spray Device, which is Described in the Present Invention, to Human Volunteers, Compared to a Parenteral Commercial Influenza Vaccine

The open randomised clinical study was carried out in absolute accordance with the principles of the declaration of Helsinki and with the local laws and directives as regards clinical studies. After the protocol had been accepted by the ethics committee of the canton of Lucerne and the Swiss Federal Health Authority had been notified, 80 healthy volunteers (18-64 years old) gave their consent in writing to participate in the test. Volunteers were excluded from the test if they had signs of an acute or chronic disease at the time of the immunisation or if they were treated with immunosuppressive medicaments at the same time of if they had a known immunodeficiency.

The intranasal vaccine formulations were administered to each of three groups consisting of 20 volunteers (Table 1). Groups A and B were given on day one 2 doses of the formulations A and B, respectively, in each nostril and 2 doses one week later. Group C were given on day one 2 doses of a double-concentrated formulation A. Group D were vaccinated intramuscularly in the deltoid region with the parentaral formulation. Immediately before the first vaccination and one month after the first immunization (day 29±2) blood and saliva samples (Omnisal®, GB) were taken. Due to a technical problem, only the saliva samples of the first 47 patients could be assessed. The brush cytology of the nasal cavities was carried out before the immunization and on days 4, 8 and one month after the first immunization.

The blood and saliva samples were coded for the analyses.

The serum immune response to the HA vaccine component was determined by means of a standard test using four hemagglutinin units of the corresponding antigens. Before the test, the sera were treated for 30 min at 56° C. The titres are expressed as reciprocal value of the highest dilution of the serum which completely inhibited hemagglutination. A titre of ≧1:40 was considered to be protective.

Overall and influenza specific IgA antibodies Were determined by means of conventional ELISA techniques (Tamura, S. I., Ito, Y., Asanuma, H. et al., Cross-protection against influenza virus infection afforded by trivalent inactivated vaccines inoculated intranasally with cholera toxin B subunit. J. Immunol. 149 (1992) 981-987). The virus-specific IgA values were expressed as ELISA units of specific IgA per μg of the overall IgA concentration.

The nasal epithelial cells were collected exclusively from the Maxillo turbinates of both nasal cavities in every person using the same type of a small nylon brush that was used in the cytopathologic examinations during the bronchoscopy (Glück, U., Gebbers, J. -O., Nasal cytopathology in smokers: a possible biomarker of air-pollution? Am. J. Rhinol. 10 (1996) 55-57). The collection of the samples was carried out by the same analyser (U. G.) under rhinoscopic control with a rotating and translational movement along the lower turbinate attachment. The cells were transferred to a glass object carrier and fixed immediately with a solution of 200 ml ethanol +100 ml acetone +6 drops of trichloroacetic acid.

The object carriers dyed with Papanicolaou were analysed by pathologists who were trained in cyopathology at the Institute of Pathology in the canton's hospital of Lucerne and who were not informed on the vaccination state.

The average cell numbers of ciliated cells, goblet cells, lymphocytes, centroblasts, neutrophils, eosinophils and squamous epithelial cells were determined in 25 representative regions per object carriers at 100×.

The significance between the baseline and the post-stimulation titres was determined by means of the paired-T test. Differences in the capability of the four vaccinations to trigger anti-HA protection antibodies in the studied group was determined by χ².

All disadvantageous events observed during the clinical study had to be recorded. A disadvantageous event was defined as a disadvantageous change of the baseline (pre-vaccination) state of the persons, regardless of whether the event was considered to be in connection with the vaccination or not. Any disadvantageous events (local or systemic reactions) occurring after the immunization were recorded by the clinician on a special report form for disadvantageous events. The baseline rate of disadvantageous events was determined prior to the immunization.

For the study, 80 persons having an average age of 40 years and a comparable social status were recruited. 27.5% of the participants were female. All 3 nasal vaccination preparations as well as the parenteral vaccines were tolerated well. As regards anamnesis, there was no significant difference between the 3 nasal vaccine substance groups and all 3 formulations were tolerated well. In few cases only, the following potential accompanying reactions were reported: fever, tiredness, sickness, rhinitis, blocked nose and rhinopharyngitis. The parenteral virosomal vaccine, too, was tolerated very well.

The serologic immune response is shown in Table 2. A significant increase in the titre was measured in group A (2 nasal vaccinations at an interval of 7 days), group C (1 nasal vaccination, double dose) and in group D (parenteral vaccination against all 3 virus strains). The highest geometric means of the antibody titre (GMT) were found in groups A and D. Group D significantly had the highest GMT values against the H1N1-strain (p≦0.05). As regards the H3N2-strain, there was no significant difference between the groups A and D.

These groups reacted significantly better than groups B and C. As regards the B-strain, there was no significant difference in groups A, C and D. These groups, however, had significantly higher titres than group B. The serum conversion rates were at the highest in groups A and D. Usually, they were significantly higher than the rates in groups B and C and, for all 3 strains, they fulfilled the serologic requirements as regards parenteral influenza vaccines in accordance with the European Community (Commission of the European Community, Directives for medical products in the European Community. Harmonisation of the requirements for influenza-vaccines. (1992) p. 93-98. Luxemburg: European Community Publication Office).

The humoral mucosal immune response (saliva) is shown in Table 3. The highest increase in the IgA titre was found in group A. This increase was significantly better than in the other groups. If the overall IgA is taken into consideration, the GMT in group A was also the highest. The mucous conversion rate (four-fold increase in the IgA titre), too, was significantly the highest in group A. In the case of intramuscular vaccination, the mucous conversion rates were very low.

The brush cytology of the nasal mucosa was carried out in groups A, B and C. The results are shown in FIG. 5. We determined the number of cells of the nasal mucosa epithelium (ciliated and non-ciliated columnar cells, goblet cells and squamous epithelial cells) and of the cells of the myelo/mono- and lymphopoiesis (lymphocytes, eosinophils, neutrophiles and centroblasts). In group A, on day 4 and day 8 after the first vaccination, a significant hyerplasia of goblet cells could be detected. Furthermore, a strong increase in lymphocytes and centroblasts could be observed on the same days. In addition, an increase in eosinophils and neutrophiles could be observed on day 8 after the first initial vaccination. The number of columnar cells remained unchanged.

In group B, there was only a slight increase in lymphocytes, neutrophile and eosinophile granulocytes. There was no sign of activated lymphocytes in this group.

In group C, an even stronger hyerplasia of goblet cells than in group A was observed. Furthermore, in this group the largest increase in eosinphile and neutrophile granulocytes was on day 4 and day 8. In this group, the increase in lymphoblasts was smaller than in group A. p=≦0.05. One month after the first vaccination, the previous state as regards the composition of cells was reached again in all of the groups.

FIG. 5 relates to the nasal cytology: quantification of the different cell populations which have been obtained from the persons (20 of each group) by nasal smear technique up to 29 days after three kinds of intranasal vaccination. The significant increase in centroblasts is to be regarded as a sign of a local immune response in group A in contrast to the significantly small increase in groups B and C (p<0.005).

### EXAMPLE 8

Comparison of the Clinical Effectiveness of Intranasal Virosomal Influenza Vaccines with HLT or PCG in Human Volunteers, Administered in a Novel Spray Device Which has been Described in the Present Invention

The immune response and tolerability of two anti-influenza spray vaccines were examined in a double-blind experiment which was carried out with, all in all, 158 healthy Swiss volunteers aged 18 to 67.

A trivalent virosomal influenza vaccine (purified HA formulated with phosphatidyl choline) was combined with heat labile toxin (HLT) of E. coli in a formate suitable for intranasal application. A human dose contained 7.5 μg HA of each influenza strain and 2 μg of HLT. On days 1 and 8, one dose per nostril was administered to groups of individuals aged 18 to 59 or ≧60. Approximately four weeks after the immunization, serum samples were taken. The reactions were rare and weak. The percentage of persones (adults or elderly) reaching the protective serum-anti-HA antibody titre (≧40) was as follows: A/Bavaria (92%, 91%), A/Wuhan (92%, 78%) and B/Beijing (59%, 50%). The GMT after the immunization and the multiple of the increase of GMT were comparable with regard to the two age groups. The percentage of people (adults or elderly) who had the non-protective baseline titre but The results show that a virosomal influenza vaccine containing a mucosal adjuvant which is administered intranasally can induce a high mucosal antibody reaction in mice.

PRE-CLINICAL STUDY OF THE VIROSOMAL INFLUENZA VACCINE
IN MICE: INTRANASAL APPLICATION
(Table 5 of Example 9)

| | Group 1 | Group 2 | Group 3 | Group 4 | Group 5 | Group 6 |
|---|---|---|---|---|---|---|
| Day 0 | infl. − | infl. − | infl. − | infl. − | influenza- | influenza- |
| | viros. + | viros. + | viros. + | viros. + | virosomes | virosomes |
| | HLT i.n. | HLT i.n. | PCG i.n. | PCG i.n. | | |
| Day 7 | infl. − | — | infl. − | — | infl.-Virus | — |
| | viros. + | | viros. + | | i.n. | |
| | HLT i.n. | | PCG i.n. | | | |
| Day 28 | NW & BAL | NW & BAL | NW & BAL | NW & BAL | NW & BAL | NW & BAL |

IgA-influenza-antibodyin mice
GMT of the reciprocal titre (ELISA)
(Table 6 of Example 9)

| | H1N1 | H1N1 | H3N2 | H3N2 | B | B |
|---|---|---|---|---|---|---|
| Groups | NW | BAL | NW | BAL | NW | BAL |
| 1 | 320 | 12800 | 380 | 3200 | 320 | 9220 |
| 2 | neg. | 140 | neg. | 24 | neg. | 1540 |
| 3 | 160 | 760 | 470 | 760 | 220 | 770 |
| 4 | neg. | neg. | neg. | neg. | 10 | neg. |
| 5 | 4 | neg. | neg. | neg. | 4 | neg. |
| 6 | neg. | neg. | neg. | neg. | neg. | neg. |

### EXAMPLE 10

Clinical Assessment of a Mucosal Hepatitis B (HBs) Vaccine

The vaccine was produced according to Example 4 and filled in a nose spray applicator according to Example 5. The product was tested with 10 volunteers: 100 μl were applied into each nostril on day 1 and again one week later. Blood samples were taken on day 1 (before the vaccination) and on day 29. The anti-HBs-antibodies were determined via RIA (Abbott). The geometric mean of the titre before the vaccination was 7 IU/ml and 159 IU/ml on day 29.

### EXAMPLE 11

Mucosal Immune Response of Mice after Intranasal Application of a DNA Plasmid Coding for the HN Antigen of Mumps Virus Introduced in Influenza Virosomes Containing 10% Cationic Phosholipids and Mixed with HLT (5 μg/ml)

We immunised groups of mice intranasally with a) naked DNA coding for the HN antigen of the mumps virus (Group C) or b) in DNA encapsulated in virosomes after pre-immunisation with virosomes (Group A) or c) without pre-immunisation (Group B). A control group (H) was immunised intranasally with living Urabe mumps virus (Priorix, SKB Rixensart). As shown in Table 7, the geometric mean of the titre GMT) of the IgG in the group of mice containing the pre-immunisation (A) was higher than the IgG described in Groups B and C of the mice. (Lovell G H: Proteosomes, hydrophobic anchors, iscoms and liposomes for improved presentation of peptide and protein vaccines. In: New Generation Vaccines (1990) (G. C. Woodrow and M. M. Levine, eds.) Dekker, New York, p. 141-168; Cusi, M. G. and Glück, R.: Intranasal immunization of mice with mumps DNA entrapped into influenza virosomes. IBC's 4^{th} Annual Conference on Genetic Vaccines, Oct. 25-27, 1998, Washington D.C.). The group of mice which had been immunised with naked DNA intranasally developed a very low IgG level, whereas the mice immunised with the mumps virus intranasally (Group H) showed a good IgG response. During analysis of mucosal immunity we found that all the groups of mice, apart from the one immunised with naked DNA, developed IgA. Only in the nose washs (NW) of the mice immunised with the mumps virus intranasally could we detect an increased titre of IgA (Group H).

Cytokin was measured using primary spleen cells from mice spleens removed twelve days after immunisation. Table 8 summarises representative measuring data obtained during two separate experiments. Cells which had been stimulated with mumps virus antigen from mice which had been vaccinated (i. n.) with DNA virosomes induced the production of IL-2 and IFN-γ. Also, mice infected with influenza induced the production of IL-4. Cells taken from the animals immunised with the mumps virus produced IFN-γ, IL-2, IL-4 and IL-10 after in vitro stimulation with Mumps antigen. The immunisation with DNA virosomes such as the control immunisation with the purified mumps antigens correlated with the Th1 phenotype. Also, taking into consideration the ratio of the IgG total level and the virus-specific IgG1 or IgG2a, the amount of IgG2a isotype in Group A dominated, which shows a Th2 response.

TABLE 7
The geometric mean of the titre (GMT) of humoral IgG, IgA in the
bronchoalveolar rinse (BAL) and IgA in the nosewash in mice

| mice | IgG | BAL IgA | NW IgA |
|---|---|---|---|
| Group A | 356 | 8 | 10 |
| Group B | 15 | 11 | 10 |
| Group C | 12 | 2 | 2 |
| Group H | 1585 | 2 | 20 |

TABLE 8
Representative measuring data of the cytokin production in mice, obtained
during two separate experiments

| Group | IL-2 | IFN-y | IL-4 | IL-10 |
|---|---|---|---|---|
| A | 300 | 300 | 150 | 0 |
| B | 150 | 625 | 0 | 0 |
| C | 300 | 100 | 0 | 0 |
| D | 600 | 100 | 150 | 600 |

### EXAMPLE12

Treatment of Blocked Nose in Volunteers via Influenza Virosomes with and without HLT in a New Spray Applicator

In a clinic, 30 volunteers with cold symptoms and acute blocked nose were selected. The breathing intensity of all volunteers was measured in each individual nostril rhinomanometrically (in Pascal). Subsequently, they were randomised and divided into 3 groups of 10. Group A were administered a dose of the vaccine (100 μl in each nostril) according to Example 1, Group B were administered the same dose but without the mucosal adjuvant HLT, Group C were administered 100 μl 0.9% NaCl (physiological saline) in each nostril. The air streams were measured 15 min, 30 min, 1 hour and 2 hours later.

The measuring data are summarised in Table 9. Both in Group A and B, the values were significantly better than in Group C. In both Groups, a clear improvement of the breathing functions could be observed.

TABLE 9
Rhinomanometric geometric means in volunteers before and after spray
treatment

| | breathing intensity in Pascal | | | | |
|---|---|---|---|---|---|
| Group | 0 min | 15 min | 30 min | 60 min | 120 min |
| A | 490 | 693 | 712 | 681 | 521 |
| B | 497 | 687 | 705 | 672 | 538 |
| C | 488 | 497 | 513 | 521 | 501 |

### EXAMPLE 13

Treatment of Mucosa Lesion in Volunteers via Influenza Virosomes and HLT

Volunteers were vaccinated intranasally, as described in Example 7. As described herein, the average goblet cells were determined. 3, 7 and 28 days after the intranasal vaccination. We were able to determine epithelial changes with goblet cell hyperplasia in cytological smears (Glück, U., Gebber, J. -O: Nasal cytopathology in smokers: a possible biomarker of air pollution? Am. J. Rhinol. 10 (1996) 55-57). Goblet cells have a protective function for the mucosal sayer. One month after the first nasal vaccination, the cellular state of the mucosa was significantly better in comparison with the previous state.

This new vaccine can, thus, be used as therapeutic agent for the treatment of damaged nasal mucosa.

### EXAMPLE 14

Prevention of enterotoxic E. coli Diarrhoe via Nasal Application of HLT with and without Influenza Virosomes

A group of travellers who had visited Tunisia consisted of 38 people. All the volunteers gave their consent to participate in the study. After randomisation, 19 volunteers were vaccinated with the preparation according to Example 1 twice with a week in-between, whereas 19 subjects were not administered a vaccine. 28 days after the first intranasal vaccination, blood samples were taken from all the volunteers. The 19 people vaccinated exhibited a high IgG level in the serum against the mucosal adjuvant (HLT). The control group remained anti-HLT-negative. Before leaving Tunisia, one month later, a specific form regarding occurrences as to health was distributed to all participants. The group returned from Tunisia 20 days later and said forms were analysed with regard to diarrhea diseases.

In the group of the vaccinated subjects, only two people reported diarrhea problems, whereas in the non-vaccinated group 9 people suffered from diarrhea during their stay in Tunisia. These data show that the vaccine is also effective as to the prevention of diarrhoe of the enterotoxic E. coli disease.

### EXAMPLE 7

TABLE 1
Clinical protocol

| | | | | | composition | |
|---|---|---|---|---|---|---|
| | | | | | in μg | composition |
| | | | | | complete | in μg |
| | | average | number of | | vaccination: | complete |
| vaccine- | N | age in | vaccinations | | HA per | vaccination: |
| group | (male) | years | (time in-between) | application | strain | HLT |
| A | 20 (14) | 39.7 | 2 (1 week) | intranasally | 15 | 2 |
| B | 20 (14) | 35.5 | 2 (1 week) | intranasally | 15 | — |
| C | 20 (16) | 43.8 | 1 | intranasally | 15 | 2 |
| D | 20 (14) | 41.2 | 1 | intra- | 15 | — |
| | | | | muscular | | |

1. A vaccine for intranasal application consisting of:
(a) influenza surface proteins which a formulated in a liposomal way (virosomes);
(b) a mucosal adjuvant of bacterial origin;
(c) a specific spray applicator which is constructed in such a way that almost 100% of a sprayed dose can be fully applied to the nasal mucosa that is important with regard to effectiveness.

2. The vaccine according to claim 1, wherein the influenza surface protein is hemagglutinin HA.

3. The vaccine according to claim 1 or 2, wherein the influenza surface proteins are hemagglutinin HA and neuraminidase NA.

4. The vaccine according to any one of claims 1 to 3 which contains further antigens.

5. The vaccine according to claim 4, wherein the other antigens are bound to the surface of the virosomes.

6. The vaccine according to claim 4 or 5, wherein the antigens are antigens of pathogenic organisms.

7. The vaccine according to claim 6, wherein the pathogenic organism is a virus, a bacterium, a fungus or a parasite.

8. The vaccine according to claim 7, wherein the virus, the bacterium, the fungus or the parasite is selected from the group consisting of: hepatitis A virus, hepatitis B virus, respiratory syncytial virus (pneumovirus), parainfluenza virus, mumps virus, Mobilli virus, HIV, diphtheria bacillus (Corynebacterium diphtheriae), tetanus bacillus (Clostridium tetani), pneumococci, Haemophilus influenzae, E. coli, Candida albicans, Candida tropicalis, Candida pseudotropicalis, Candida parapsilosis, Candida krusei, Aspergillus species, Trichomonas species, Trypanosoma species, Leishmania species, Toxoplasma gondii, Plasmodium falciparum, Plasmodium vivax, Plasmodium ovale, Plasmodium malariae, Trematoda species and Nematoda species.

9. The vaccine according to any one of claims 1 to 8 which is an influenza vaccine.

10. The vaccine according to any one of claims 1 to 9 which may be used for preventing and/or treating general infections/infectious diseases.

11. The vaccine according to any one of claims 1 to 10 for preventing and/or treating influenza diseases.

12. The vaccine according to claim 10 or 11 for preventing and/or treating a blocked nose.

13. The vaccine according to any one of claims 10, 11 or 12 for treating injuries of the nasal mucosa.

14. The vaccine according to any one of claims 1 to 13, wherein the hemagglutinin content per dose (100 μl) ranges from 1-30 μg, preferably from 3-10 μg most preferably, the content is 3.75 μg.

15. The vaccine according to any one of claims 2 to 14, wherein the ratio of liposomes-phospholipid to HA is between 1:10 and 20:1.

16. The vaccine according to claim 15, wherein the ratio of liposomes-phospholipid to HA is between 1:1 and 10:1.

17. The vaccine according to claim 15 and/or 16, wherein the ratio is 3:1.

18. The vaccine according to any one of claims 15 to 17, wherein the phospholipid of the liposomes is selected from the group of neutral, cationic and anionic phospholipids.

19. The vaccine according to any one of claims 1 to 18, wherein the mucosal adjuvant is an active toxin, an inactive toxin and/or a non-toxic derivative thereof.

20. The vaccine according to claim 19, wherein the toxin and/or the non-toxic derivative thereof is heat labile toxin (HLT), cholera toxin (CT) and/or procholera genoid (PCG).

21. The vaccine according to any one of claims 1 to 20 which contains the heat labile toxin (HLT) and/or the cholera toxin (CT) in a ratio that ranges from 1:2 to 20:1, preferably from 1:1 to 1:10 and that, most preferably, is 7.5:1.

22. The vaccine according to claim 19 or 20 which contains the heat-inactivated procholera genoid (PCG), the HLT inactivated by means of recombination technology and/or cholera toxin (CT) as mucosal adjuvant, wherein the ratio of HA:PCG (and/or rHLT, and/or rCT) is between 3:1 and 1:20.

23. The vaccine according to claim 22, wherein the ratio of HA:PCG (and/or rHLT, and/or rCT) is between 1:1 and 1:10.

24. The vaccine according to claim 23, wherein the ratio HA:PCG (and/or rHLT, and/or rCT) is 1:2.

25. The vaccine according to any one of claims 1 to 24 which contains, in addition, the DNA and/or RNA plasmids that are bound to and/or in virosomes.

26. The vaccine according to any one of claims 1 to 25 which is an influenza vaccine.

27. The mucosal adjuvant according to any one of claims 1 to 26 which is the heat labile toxin (HLT) that is eluted from E. coli with a lactose buffer according to Example 2 and purified on a chromatography column with lactosyl gel (Pharmacia).

28. A collar (24) with a connection element (28) for placing on an application device (2), in particular for spraying a pharmaceutically active substance according to any one of claims 1 to 27, and a support means (30), wherein the connection element (28) and the support means (30) are placed in such a way that they form an optimum angle of spraying (α) for dispensing the substance with the application device.

29. The collar (24) according to claim 28, wherein the angle of spraying (α) is between 50° and 80° to the horizontal line.

30. The collar (24) according to claim 28 or 29, wherein the connection element (28) is a substantially cylindrical tubular section.

31. The collar (24) according to claim 30, wherein the tubular section (28) may be put over at least a part of the nose piece (20) of the application device (2).

32. The collar (24) according to any one of claims 28 to 31, wherein the support means (30) at the user's end is curved in such a way that it essentially corresponds to the form of an outer section of the user's upper lip.

33. The collar (24) according to, any one of claims 28 to 32, wherein the collar (24) is fixed to the application device (2) in such a way that it cannot rotate.

34. The collar (24) according to claim 33, wherein the tubular section (28) and the part of the nose piece (20) over which the tubular section (28) can be put have, at least in part, a polygon form in order to form a locking means.

35. An application device (2), in particular for spraying a pharmaceutically active substance according to any one of claims 1 to 27 with a pump element (6), a finger collar (10) for activating the pump element (6), a connection element (8) for a tight connection with a storage container (4) and a nose piece (16) having a first section (18) and a second section (20), wherein the two sections (18, 20) of the nose piece (16) are formed in such a way that a spray outlet (14) of the nose piece (16) essentially extends to the main nasal cavities of a patient.

36. The application device (2) according to claim 35, wherein at least the second section (20) of the nose piece (16) essentially has a cylindrical form.

37. The application device (2) according to claim 36, wherein the diameter of the second section (20) ranges between 3 mm and 10 mm, preferably is approximately 7 mm.

38. The application device (2) according to any one of claims 35 to 37, wherein the second section (20) of the nose piece (16) has a length of 5 to 50 mm, preferably of approximately 20 mm.

39. The application device (2) according to any one of claims 35 to 38, wherein the first section (18) of the nose piece (16) has a length of 10 to 40 mm, preferably of approximately 30 mm.

40. The application device (2) according to any one of claims 35 to 39, wherein the distance between the finger collar (10) and the spray outlet (14) which is to be located at the top end is at least 30 mm, preferably approximately 45 mm.

41. The application device (2) according to any one of claims 35 to 40 with a collar (24) according to any one of claims 28 to 34.

42. The application device (2) according to claim 41 which is made in one piece or integrally connected with the collar (24).
